(19) United States
(12) Patent Application Publication
(10) Pub. No.: US 2026/0263539 A1
(43) Pub. Date: Sep. 10, 2026

(54) COMPOSITION FOR USE

(71) Applicant: SOCIÉTÉ DES PRODUITS NESTLÉ S.A., Vevey (CH)

(72) Inventors: Shaillay Kumar DOGRA, Epalinges (CH); Olga SAKWINSKA, Pully (CH); Laurent FERRIER, Servion (CH)

(21) Appl. No.: 19/166,783

(22) PCT Filed: Mar. 28, 2024
(86) PCT No.: PCT/EP2024/058601

§ 371 Date: Sep. 18, 2025

(30) Foreign Application Priority Data
Mar. 30, 2023 (EP) 23165376.7

(51) Int. Cl. A61K 35/745; A61K 35/00; A61K 35/744; A61K 47/26; C12Q 1/04; C12Q 1/14

(52) U.S. Cl. CPC A61K 35/745; A61K 35/744; A61K 47/26; C12Q 1/04; C12Q 1/14; A61K 2035/115

(57) ABSTRACT

The present invention provides methods for identifying an infant or young child at risk of stunted growth and compositions for use in preventing and/or treating stunted growth in an infant or young child.

### FIELD OF THE INVENTION

The present invention relates to methods for identifying an infant or young child at risk of stunted growth and compositions for use in preventing and/or treating stunted growth in an infant or young child.

### BACKGROUND TO THE INVENTION

Investigation of the impact of malnutrition on growth has focused primarily on weight-related outcomes. However, approaches that aim to identify and improve reduced weight gain may have negative long-term consequences; for example the gain in weight may be associated with a risk of metabolic disorders.

Several studies have investigated the issue of undergrowth by using microbiome directed solutions (for example Chen R Y et al. NEJM 2021; 384:1517-28; Subramanian S, et al. Nature 2014; 510 (7505): 417-421). However, most of these studies report gain in weight-for-age or weight-for-height which could be related to the use of calorie rich foods in these interventional trials, and potentially have consequences such as subsequent risk of metabolic disorders.

There is a need for further approaches and methods to identify, and/or treat or prevent, height or length related outcomes of reduced growth, for example as a result of malnourishment, in infants or young children.

### SUMMARY OF THE INVENTION

The present invention is based, at least in part, on the inventors' surprising determination that reduced levels of Bifidobacterium pseudocatenulatum (B. pseudocatenulatum) and/or Streptococcus thermophilus (S. thermophilus) in the gut microbiota are associated with linear growth faltering in a cohort of infants and young children. Further, the inventors have determined that Bifidobacterium pseudocatenulatum can be promoted by human milk oligosaccharides (HMOs). To the best of the inventors' knowledge, the relationship between linear growth faltering and the microbiome have not previously been investigation, or where they have no positive results have been identified. For example, either no association or improvement of height-for-age z (HAZ) score or length-for-age z (LAZ)-score was determined (Subramanian S, et al. Nature. 2014; 510 (7505): 417-421) or LAZ correlation with a few bacteria only of duodenal microbiota was reported (Chen R Y, et al. N Engl J Med. 2020; supra).

Accordingly, the present invention provides approaches for identifying an infant or young child at risk of stunted growth by determining the abundance of Bifidobacterium pseudocatenulatum and/or S. thermophilus in one or more samples obtained from the infant. The invention also provides approaches for preventing and/or treating stunted growth in an infant or young child by promoting Bifidobacterium pseudocatenulatum and/or S. thermophilus in the gut microbiota. The invention may thus comprise promoting the abundance and/or activity of Bifidobacterium pseudocatenulatum and/or S. thermophilus in the gut microbiota.

Thus, in a first aspect the present invention provides a method for identifying an infant or young child at risk of stunted growth, wherein said method comprises determining the abundance of Bifidobacterium pseudocatenulatum and/or S. thermophilus in one or more samples obtained from the infant.

Suitably, the method comprises determining the abundance of Bifidobacterium pseudocatenulatum in one or more samples obtained from the infant.

Suitably, an infant or young child with reduced levels of B. pseudocatenulatum is identified as at risk of stunted growth.

The stunted growth may be stunted height or length. Suitably, stunted height or length may be defined as a reduced length-for-age z-score (LAZ) or a reduced LAZ over time. Suitably, stunted height or length may be defined as a reduced hength-for-age z-score (HAZ) or a reduced HAZ over time.

In another aspect the present invention provides a composition for use in preventing and/or treating stunted growth in an infant or young child, wherein the composition promotes B. pseudocatenulatum in the gut microbiota of the infant or young child.

The composition may comprise a B. pseudocatenulatum microorganism. Suitably, the composition comprising a B. pseudocatenulatum microorganism may be administered in combination with a prebiotic.

The composition may comprise a prebiotic. Suitably, the composition comprising a prebiotic may be administered in combination with a B. pseudocatenulatum microorganism.

The present invention further provides a combination of a B. pseudocatenulatum microorganism and a prebiotic for use in preventing and/or treating stunted growth in an infant or young child.

The prebiotic may be in the form of a dietary or nutritional composition. The prebiotic may comprise a human milk oligosaccharide (HMO).

The invention further provides a HMO, or a combination of HMOs, for use in preventing and/or treating stunted growth in an infant or young child, wherein the HMO or combination of HMOs promote B. pseudocatenulatum in the gut microbiota of the infant or young child.

The HMO may be selected from the group consisting of 2′-FL, 3-FL, di-FL, 3′-SL, 6′-SL, LNT and LNnT, and any combination thereof. The HMO may be any HMO, or combination of HMOs, as defined herein.

The HMO may be selected from the group consisting of 2′-FL, di-FL, 6′-SL and LNnT, and any combination thereof. The HMOs may be a combination of 2′-FL and di-FL. The HMOs may be a combination of 6′-SL and LNnT.

The HMO(s) may be provided in combination with galacto-oligosaccharides (GOS).

The present invention also provides a B. pseudocatenulatum microorganism for use in preventing and/or treating stunted growth in an infant or young child.

The treatment and/or prevention of stunted growth may be associated with enhanced bone development and/or bone strength in the subject.

The invention further provides a method of preventing and/or treating stunted growth in an infant or young child, said method comprising administering a composition which promotes B. pseudocatenulatum in the gut microbiota to the infant or young child.

In a further aspect, the present invention provides the use of a B. pseudocatenulatum microorganism and/or a prebiotic as defined herein in the manufacture of a medicament for preventing and/or treating stunted growth in an infant or young child.

The invention also provides the use of a composition for modulating the abundance of B. pseudocatenulatum in the gut of an infant or young child. The composition may be any composition as defined herein.

The invention further provides a probiotic composition comprising B. pseudocatenulatum.

The invention also provides a synbiotic composition comprising B. pseudocatenulatum and a prebiotic. The prebiotic may be a prebiotic as defined herein.

Suitably, for any aspect of the invention S. thermophilus may be provided as an alternative to B. pseudocatenulatum.

Suitably, any aspect of the invention may be directed to a combination of B. pseudocatenulatum and S. thermophilus.

### DESCRIPTION OF DRAWINGS

FIG. 1—Schematic of Microhealth study

FIG. 2—Microbiome profiling of populations distinguished by difference in dynamic changes in LAZ scores over time. Q1 is infants with negative slopes of length-for-age z (LAZ) scores over time, which are defined as ‘undergrowth’. Q4 is infants with positive slopes of LAZ scores over time, which are defined as ‘reference’.

FIG. 3—Bifidobacterium pseudocatenulatum was identified as a bacterial signature of inadequate height profile up to 24 months using both (A) dynamic changes and (B) static outcomes.

FIG. 4—Streptococcus thermophilus was identified as a bacterial signature of inadequate height profile up to 24 months using both (A) dynamic changes and (B) static outcomes.

FIG. 5—Illustrative WHO height-for age (Z-scores) for girls aged 2 to 5 years.

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range.

The publications discussed herein are provided solely for their disclosure prior to the filing date of the present application. Nothing herein is to be construed as an admission that such publications constitute prior art to the claims appended hereto.

The methods and systems disclosed herein can be used by doctors, health-care professionals, lab technicians, infant or young child care providers and so on.

### Stunted Growth

The UNICEF and WHO definition of stunting, or stunted growth, refers to a child who is too short for his or her age. These children can suffer severe irreversible physical and cognitive damage that accompanies stunted growth. The effects of stunting can last a lifetime and even affect the next generation (https://data.unicef.org/topic/nutrition/malnutrition). In 2020, there were about 22% children under 5 years of age affected by stunting, as per UNICEF/WHO/World Bank Joint Child Malnutrition Estimates, 2021 Edition. This equates to 149.2 million stunted children under 5 years of age. Stunted growth is seen in infants and toddlers but goes beyond to pre-school and school-age children (Leroy J L, Ruel M, Habicht J P, Frongillo E A. J Nutr. 2014; Dutta A, et al. Food Nutr Bull. 2009).

Stunted growth in infants and young children may be caused by many factors, from poor in utero environment, Aflatoxin exposure, intergenerational effects, environmental enteric dysfunction, lack of nutrition and dietary diversity, infections (such as Campylobacter infection), diarrhea, water, sanitation, and hygiene issues and other causes such as maternal factors. Several studies have investigated the issue of undergrowth by using microbiome directed solutions (for example Chen R Y et al. NEJM 2021; Subramanian S, et al. Nature 2014). However, most of these studies report gain in weight-for-age or weight-for-height which could be related to use of caloric rich foods in these interventional trials, and potentially have consequences such as subsequent risk of metabolic disorders. To the best of the inventors' knowledge, the relationship between linear growth faltering and the microbiome have not previously been investigated, or where they have no positive results have been identified. For example, either no association or improvement of height-for-age z-score (HAZ) or length-for-age z-score (LAZ) was determined (Subramanian S, et al. Nature. 2014; supra) or LAZ correlation with a few bacteria only of duodenal microbiota was reported (Chen R Y, et al. N Engl J Med. 2020; supra).

As used herein, ‘stunted growth’ may refer to stunted height or length of an infant or young child.

Growth indicators such as length/height-for-age may be used to identify children who are stunted (short) due to prolonged undernutrition or repeated illness.

Suitably, the stunted growth may be defined as a reduced length-for-age z-score (LAZ) or a reduced LAZ over time. LAZ may also be referred to as height-for-age z-score (HAZ).

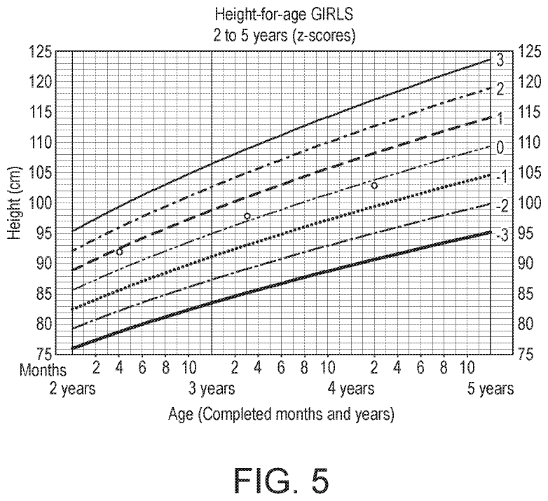

Normally growing children follow trends that are, in general, parallel to the median and z-score lines (see FIG. 5). Most children will grow in a “track”, that is, on or between z-score lines and roughly parallel to the median; the track may be below or above the median. When interpreting growth charts, the following situations are of caution, and may indicate a problem or suggest risk: a child's growth line crosses a z-score line, there is a sharp incline or decline in the child's growth line, or the child's growth line remains flat (stagnant) i.e. there is no gain in weight or length/height.

Stunting may be defined as a static outcome. For example, stunted growth may be defined as a length/height-for-age below-2. For example, stunted growth may be defined as cases with height-for-age less than-2 standard deviations from the median of the World Health Organization (WHO) Child Growth Standards (https://sdgdata.gov.uk/2-2-1/#:~:text=Definitions,(WHO)%20Child %20Growth %20Standards).

Stunted growth may be defined as a dynamic change, for example a change in LAZ assessed over time (e.g. 0-36 months, 6-36 months or 6-24 months). Infants with a decline in LAZ scores over time may be defined as suffering from stunted growth.

Dynamic changes in growth may be referred to a “growth velocity” or “height velocity”. Growth or height velocity may be defined as shown in Equation (1).

$$V = \frac{H 2 - H 1}{Δ t} \qquad ( 1 )$$

wherein H1 and H2 are the two height measurements, and Δt is the time interval between measurements. The stunted growth may be associated with malnourishment.

Malnutrition results from decreased food consumption and/or illness. It is associated with greater risk of medical complications and infections, increased risk of death from illness and infections, and micronutrient deficiencies. Non-limiting examples of micronutrient deficiencies associated with malnutrition are iron deficiency, iodine deficiency, and vitamin A deficiency. The most common way to assess malnutrition, particularly in infants and young children, is through anthropometric measurements. It is usually diagnosed in one of three ways: by weighing a subject and measuring the subject's height; by measuring the circumference of the subject's mid-upper arm (MUAC); and/or by checking for oedema in the subject's lower legs or feet. Malnutrition may be divided into two types: severe acute malnutrition (SAM) and moderate acute malnutrition (MAM). A subject is classified as having SAM if the subject's weight-for-height Z-scores (WHZ) is below three standard deviations (−3 s.d.) from the median of the World Health Organization (WHO) reference growth standards. A subject with a WHZ between −2 s.d. and −3 s.d. from the median of the WHO reference growth standards is categorized as having MAM. If a subject is between about six months and about five years of age, a MUAC measurement of less than 12.5 cm also indicates that a subject is suffering from moderate acute malnutrition. Finally, the presence of oedema in both feet and lower legs of a subject is a sign of SAM. WHO reference growth standards are available from the WHO. See for example, World Health Organization Department of Nutrition for Health and Development: WHO child growth standards growth velocity based on weight, length and head circumference: methods and development; World Health Organization, 2009, or the current edition.

Suitably the infant or young child may be suffering from SAM. Suitably, the infant or young child may be suffering from MAM.

A subject at risk of stunted growth may be a subject living in a geographic area with limited or no access to a comprehensive, nutritional diet and/or a subject living in a geographic area that is experiencing a disease outbreak. A subject at risk of stunted growth may be a subject that is malnourished.

Treating a subject at risk of stunted growth may reduce the occurrence or prevent stunted growth in the subject.

The term “prevention” in connection with stunted growth may refer to reducing or eliminating stunted growth.

For example, preventing stunted growth may involve reducing in treated subjects: the incidence or development of stunted growth; the development, duration, and/or severity of stunted growth, if the stunted growth does develop; or a combination thereof. For each aspect, the amount of reduction may each be about 2%, 5%, 10%, 20%, 30%, 40%, 50%, 60%, 70%, 80%, 90%, 95%, or about 100% in treated subjects, as compared to untreated subjects.

### Infant or Young Child

Suitably, the infant or young child may be less than about 60 months of age. For example, the infant or young child may be less than 48 months, less than 36 months of age or less than 24 months of age.

Suitably, the infant or young child may be from about 1 to 60 months, about 1 to 48 months, about 2 to 60 months, about 2 to 48 months, about 2 to 36 months, about 4 to 36 months, about 6 to 36 months of age or about 6 to 24 months of age.

For example, the infant or young child may be at least about 6 months, at least about 10 months, at least about 12 months, at least about 14 months, at least about 16 months, at least about 20 months, or at least about 24 months.

Suitably, the infant or young child may be about 6 to about 60 months of age, about 6 to about 48 months of age, about 6 to about 36 months of age or about 6 to about 24 months of age.

The infant may be a child under the age of 12 months. The “young child” may be a child aged between one and less than five year, or between one and less than three years.

The subject may be a mammal. Preferably, the subject is a human. Unless stated otherwise, ages referred to herein are in respect of a human subject.

### Composition

The composition may be suitable for or may suitably be administered to an infant or young child in any suitable form such as a nutritional composition in a dosage unit (for example a tablet, a capsule, a sachet of powder, etc). The composition may be in powder, semi-liquid or liquid form. The composition may be added to a nutritional composition, an infant formula, a food composition, a supplement for infant or young child, a baby food, a follow-up formula, a growing-up milk, an infant or young child cereal or a fortifier. In some embodiments, the composition of the present invention is an infant formula, a baby food, an infant or young child cereal, a growing-up milk, a supplement or fortifier that may be intended for an infant or young child.

The expressions “complementary feeding period”, “complementary period”, “transitional period”, “transitional feeding period” and “weaning period” can be interchangeably used and refer to the period during which the milk, either breast milk or formula, is substituted by other foods in the diet of an infant or young child. The infant or young child is typically moved or transitioned gradually from exclusive milk-feeding, either breast feeding or formula feeding, to mixed diet comprising milk and/or solid foods. The transitional period depends on the infant or young child but typically falls between about 4 months and about 18 months of age, such as between about 6 and about 18 months of age but can in some instances extend up to about 24 months or more. For humans, the weaning period typically starts between 4 and 6 months of age and is considered completed once the infant or young child is no longer fed with breast milk or infant formula, typically at about 24 months of age. In some embodiments, the weaning period is between 4 and 24 months.

Suitably, the composition is a dietary composition or nutritional composition.

The expressions “dietary composition” or “nutritional composition” refer to any kind of composition or formulation that provides a nutritional benefit to an individual and that may be safely consumed by a human or an animal. Said nutritional composition may be in solid (e.g. powder), semi-solid or liquid form and may comprise one or more macronutrients, micronutrients, food additives, water, etc. For instance, the nutritional composition may comprise the following macronutrients: a source of proteins, a source of lipids, a source of carbohydrates and any combination thereof. Furthermore, the nutritional composition may comprise the following micronutrients: vitamins, minerals, fibers, phytochemicals, antioxidants, prebiotics, probiotics, and any combination thereof. The composition may also contain food additives such as stabilizers (when provided in solid form) or emulsifiers (when provided in liquid form). The amount of the various ingredients can be expressed in g/100 g of composition on a dry weight basis when it is in a solid form, e.g. a powder, or as a concentration in g/L of the composition when it refers to a liquid form (the latter also encompasses a liquid composition that may be obtained from a powder after reconstitution in a liquid such as milk, water, e.g. a reconstituted infant or young child formula or follow-on/follow-up formula or infant or young child cereal product or any other formulation designed for infant or young child or young child nutrition). Generally, the nutritional composition can be formulated to be taken enterally, orally, parenterally, or intravenously, and it usually includes one of more nutrients selected from: a lipid or fat source, a protein source and a carbohydrate source. Preferably, the nutritional composition is for oral administration.

In a particular embodiment, the composition of the present invention is a “synthetic nutritional composition”. The expression “synthetic nutritional composition” means a mixture obtained by chemical and/or biological means.

The expression “infant formula” as used herein refers to a foodstuff intended for particular nutritional use by infant during the first months of life and satisfying by itself the nutritional requirements of this category of person (Article 2 (c) of the European Commission Directive 91/321/EEC 2006/141/EC of 22 Dec. 2006 on infant formulae and follow-on formulae). It also refers to a nutritional composition intended for infant or young child and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is generally given from the 6th month onwards. It constitutes the principal liquid element in the progressively diversified diet of this category of person.

The expression “baby food” means a foodstuff intended for particular nutritional use by infants or young children during the first years of life.

The expression “infant or young child cereal composition” means a foodstuff intended for particular nutritional use by infants or young children during the first years of life.

The expression “growing-up milk” (or GUM) refers to a milk-based drink generally with added vitamins and minerals, that is intended for young children or children.

The “fortifier” may be a liquid or solid nutritional composition suitable for fortifying or mixing with human milk, infant or young child formula, or growing-up milk. Accordingly, the fortifier can be administered after dissolution in human breast milk, in infant or young child formula, in growing-up milk or in human breast milk fortified with other nutrients or otherwise it can be administered as a stand-alone composition. When administered as a stand-alone composition, the milk fortifier can be also identified as being a “supplement”.

The nutritional composition may comprise a protein source. The protein can be in an amount of from 1.6 to 3 g per 100 kcal.

Protein sources based on whey, casein and mixtures thereof may be used as well as protein sources based on soy. As far as whey proteins are concerned, the protein source may be based on acid whey or sweet whey or mixtures thereof and may include alpha-lactalbumin and beta-lactoglobulin in any desired proportions.

In some embodiments the protein source is whey predominant (i.e., more than 50% of proteins are coming from whey proteins, such as 60% or 70%). The proteins may be intact or hydrolysed or a mixture of intact and hydrolysed proteins. By the term “intact” is meant that the main parts of the proteins are intact, i.e., the molecular structure is not altered, for example at least 80% of the proteins are not altered, such as at least 85% of the proteins are not altered, preferably at least 90% of the proteins are not altered, even more preferably at least 95% of the proteins are not altered, such as at least 98% of the proteins are not altered. In a particular embodiment, 100% of the proteins are not altered.

In one particular embodiment the proteins of the nutritional composition are hydrolyzed, fully hydrolyzed or partially hydrolyzed. The degree of hydrolysis (DH) of the protein can be between 8 and 40, or between 20 and 60 or between 20 and 80 or more than 10, 20, 40, 60, 80 or 90. The protein component can alternatively be replaced by a mixture or synthetic amino acid, for example for preterm or low birth weight infants.

The term “hydrolysed” means in the context of the present invention a protein which has been hydrolysed or broken down into its component amino acids. The proteins may be either fully or partially hydrolysed. It may be desirable to supply partially hydrolysed proteins (degree of hydrolysis between 2 and 20%), for example for infants or young children believed to be at risk of developing cow's milk allergy. If hydrolysed proteins are required, the hydrolysis process may be carried out as desired and as is known in the art. For example, whey protein hydrolysates may be prepared by enzymatically hydrolysing the whey fraction in one or more steps. If the whey fraction used as the starting material is substantially lactose free, it is found that the protein suffers much less lysine blockage during the hydrolysis process. This enables the extent of lysine blockage to be reduced from about 15% by weight of total lysine to less than about 10% by weight of lysine; for example, about 7% by weight of lysine which greatly improves the nutritional quality of the protein source.

In an embodiment of the invention at least 70% of the proteins are hydrolysed, for example at least 80% of the proteins are hydrolysed, such as at least 85% of the proteins are hydrolysed, or at least 90%, 95%, 98% of the proteins are hydrolysed. In a particular embodiment, 100% of the proteins are hydrolysed.

The nutritional composition may contain a carbohydrate source. This is particularly preferable in the case where the nutritional composition is an infant formula. In this case, any carbohydrate source conventionally found in infant formulae such as lactose, sucrose, saccharose, maltodextrin, starch and mixtures thereof may be used although one of the preferred sources of carbohydrates is lactose.

The nutritional composition may contain a source of lipids. This is particularly relevant if the nutritional composition is an infant formula. In this case, the lipid source may be any lipid or fat which is suitable for use in infant formulae. Some suitable fat sources include palm oil, structured triglyceride oil, high oleic sunflower oil and high oleic safflower oil, medium-chain-triglyceride oil. The essential fatty acids linoleic and a-linolenic acid may also be added, as well small amounts of oils containing high quantities of preformed arachidonic acid and docosahexaenoic acid such as fish oils or microbial oils. The fat source may have a ratio of n-6 to n-3 fatty acids of about 5:1 to about 15:1; for example, about 8:1 to about 10:1.

The nutritional composition may also contain vitamins and minerals understood to be essential in the daily diet and in nutritionally significant amounts. Minimum requirements have been established for certain vitamins and minerals. Examples of minerals, vitamins and other nutrients optionally present in the composition of the invention include vitamin A, vitamin B1, vitamin B2, vitamin B6, vitamin B12, vitamin E, vitamin K, vitamin C, vitamin D, folic acid, inositol, niacin, biotin, pantothenic acid, choline, calcium, phosphorous, iodine, iron, magnesium, copper, zinc, manganese, chlorine, potassium, sodium, selenium, chromium, molybdenum, taurine, and L-carnitine. Minerals are usually added in salt form. The presence and amounts of specific minerals and other vitamins will vary depending on the intended population. If necessary, the nutritional composition of the invention may contain emulsifiers and stabilisers such as soy, lecithin, citric acid esters of mono- and diglycerides, and the like.

The nutritional composition may also contain other substances which may have a beneficial effect such as lactoferrin, nucleotides, nucleosides, and the like.

The nutritional composition may be prepared in any suitable manner. A composition will now be described by way of example.

For example, a formula such as an infant formula may be prepared by blending together the protein source, the carbohydrate source and the fat source in appropriate proportions. If used, the emulsifiers may be included at this point. The vitamins and minerals may be added at this point, but they are usually added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. The temperature of the water is conveniently in the range between about 50° C. and about 80° C. to aid dispersal of the ingredients. Commercially available liquefiers may be used to form the liquid mixture.

The liquid mixture is then homogenised.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If the final product is to be a powder, the homogenised mixture is transferred to a suitable drying apparatus such as a spray dryer or freeze dryer and converted to powder. The powder should have a moisture content of less than about 5% by weight. The mixture may be spray-dried or freeze-dried.

If a liquid composition is preferred, the homogenised mixture may be sterilised then aseptically filled into suitable containers or may be first filled into the containers and then retorted.

The nutritional composition can be for example given immediately after birth of the infant. The nutritional composition of the invention can also be given during the first week of life of the infant or young child, or during the first 2 weeks of life, or during the first 3 weeks of life, or during the first month of life, or during the first 2 months of life, or during the first 3 months of life, or during the first 4 months of life, or during the first 6 months of life, or during the first 8 months of life, or during the first 10 months of life, or during the first year of life, or during the first two years of life or even more. In some particularly advantageous embodiments of the invention, the composition is given (or administered) to an infant or young child from about 6 months of birth of said infant or young child. For example, the composition may be given from about 6 months, about 10 months, about 12 months, about 14 months, about 16 months, about 20 months, about 24 months or about 36 months from birth.

Suitably, the composition is given (or administered) to an infant or young child from about 10 months of birth of said infant or young child.

Suitably, the composition may be administered to an infant or young child from about 6 to about 60 months of age, from about 6 to about 48 months of age, from about 6 to about 36 months of age. Suitably, the composition may be administered to an infant or young child from about 10 to about 60 months of age, from about 10 to about 48 months of age, or from about 10 to about 36 months of age.

In one embodiment the nutritional composition is given to the infant or young child as a supplementary composition to the mother's milk. In some embodiments the infant or young child receives the mother's milk during at least the first 2 weeks, first 1, 2, 4, or 6 months. In one embodiment the nutritional composition of the invention is given to the infant or young child after such period of mother's nutrition or is given together with such period of mother's milk nutrition. In another embodiment the nutritional composition is given to the infant or young child as the sole or primary nutritional composition during at least one period of time, e.g., after the 1st, 2nd or 4th month of life, during at least 1, 2, 4 or 6 months.

Suitably, the composition may comprise a probiotic comprising Bifidobacterium pseudocatenulatum.

Suitably, the composition may comprise a probiotic comprising Streptococcus thermophilus.

The term “probiotic” means microbial cell preparation or components of microbial cells with a beneficial effect on the health or well-being of the host (Salminen S, Ouwehand A. Benno Y. et al. “Probiotics: how should they be defined” Trends Food Sci. Technol. 1999:10 107-10; Hill C, et al. Nat Rev Gastroenterol Hepatol. 2014). The microbial cells are generally bacteria or yeasts.

The Bifidobacterium pseudocatenulatum may be included in the composition in, for example, an amount from about 10³ to 10¹² cfu of probiotic strain, more preferably between 10⁷ and 10¹² cfu such as between 10⁸ and 10¹⁰ cfu of probiotic strain per g of composition on a dry weight basis. In one embodiment, the Bifidobacterium pseudocatenulatum is viable. There may be both viable and inactivated Bifidobacterium pseudocatenulatum in some other embodiments.

The Streptococcus thermophilus may be included in the composition in, for example, an amount from about 10³ to 10¹² cfu of probiotic strain, more preferably between 10⁷ and 10¹² cfu such as between 10⁸ and 10¹⁰ cfu of probiotic strain per g of composition on a dry weight basis. In one embodiment, the Streptococcus thermophilus is viable. There may be both viable and inactivated Streptococcus thermophilus in some other embodiments.

The term “cfu” should be understood as colony forming unit.

Suitably, the present invention may comprise the use of a combination of a nutritional or dietary composition as described herein and a probiotic composition, as described herein e.g., a probiotic composition comprising Bifidobacterium pseudocatenulatum and/or Streptococcus thermophilus. Suitably, the present invention may comprise a synbiotic, or the use of a synbiotic. As used herein, a synbiotic may refer to a mixture comprising microorganisms and substrate(s) selectively utilized by the microorganism, preferably wherein the combination confers a health benefit on the host (see e.g. Swanson K S, et al. Nat Rev Gastroenterol Hepatol. 2020). For example, the present symbiotic may comprise HMO as a prebiotic and B. pseudocatenulatum and/or S. thermpphilus as a probiotic. Suitably, the present symbiotic may comprise HMO as a prebiotic and B. pseudocatenulatum as a probiotic

The composition for use according to the present invention may be administered by any suitable method. Preferably, the composition is for oral administration. Accordingly, the composition is preferably administered orally.

In some embodiments the composition according to the invention can be for use before and/or during the weaning period. The nutritional composition can be administered (or given or fed) at an age and for a period that depends on the needs.

Suitably, where a combination is administered to a subject (e.g., a combination of a prebiotic and probiotic as described herein), the combination may be administered separately, simultaneously or sequentially.

Suitably, the composition may be Nan Pelargon®.

Bifidobacterium pseudocatenulatum

Bifidobacterium is a genus of gram-positive, nonmotile, often branched anaerobic bacteria. They are ubiquitous inhabitants of the gastrointestinal tract, making up one of the major genera of bacteria of the gastrointestinal tract microbiota in mammals.

The genus Bifidobacterium possesses a unique fructose-6-phosphate phosphoketolase pathway employed to ferment carbohydrates. Much metabolic research on Bifidobacteria has focused on oligosaccharide metabolism, as these carbohydrates are available in their otherwise nutrient-limited habitats. Infant-associated bifidobacterial phylotypes appear to have evolved the ability to ferment milk oligosaccharides, whereas adult-associated species use plant oligosaccharides, consistent with what they encounter in their respective environments.

A type strain of Bifidobacterium pseudocatenulatum is ATCC 27919. A reference genome for Bifidobacterium pseudocatenulatum is provided by GenBank assembly accession: GCF_020541885.1.

Suitably, the Bifidobacterium pseudocatenulatum may comprise a 16S rRNA gene sequence with a cutoff identity value of 99% and minimum query and target coverages of 80% when compared to the 16S rRNA sequence of ATCC 27919 and/or GCF_020541885.1 using BLASTn. Suitable comparisons may be performed using known methods, as described by Maturana and Cárdenasm (Front Microbiol. 2021; 660920), for example.

Suitably, the Bifidobacterium pseudocatenulatum may have ANI (average nucleotide identity) of at least 95%, a TETRA (tetranucleotide frequency) of at least 0.99, and/or an AAI (average amino acid identity) of at least 95% compared to ATCC 27919 and/or GCF_020541885.1 for an entire genome dataset. Suitably, the Bifidobacterium pseudocatenulatum microorganism may have ANI (average nucleotide identity) of at least 95%, a TETRA (tetranucleotide frequency) of at least 0.99, and an AAI (average amino acid identity) of at least 95% compared to ATCC 27919 and/or GCF_020541885.1 for an entire genome dataset. Suitable comparisons may be performed using known methods, as described by Maturana and Cárdenasm (as above), for example.

Suitably, a Bifidobacterium pseudocatenulatum may be identified using a metagenomics method. Suitable metagenomics methods may be performed using shotgun sequencing data, for example. Metagenomics methods may also advantageously enable estimation of organismal relative abundance. Suitable metogenomics methods are known in the art and include MetaPhlAn 3.0, for example (see Beghini et al.; eLife 2021; 10: e65088; https://huttenhower.sph.harvard.edu/metaphlan).

In some embodiments, the Bifidobacterium pseudocatenulatum is isolated from a human.

Suitably, to ‘promote Bifidobacterium pseudocatenulatum’ means to increase the absolute or relative numbers of Bifidobacterium pseudocatenulatum in the gut microbiota. For example, a prebiotic may assist or support the growth and/or survival of the microorganism. Alternatively, a probiotic composition for use in the invention will comprise Bifidobacterium pseudocatenulatum, and thereby increase number of Bifidobacterium pseudocatenulatum within the gut microbiota.

The abundance of Bifidobacterium pseudocatenulatum in the gut microbiota may be determined, for example, by assessing the relative abundance of Bifidobacterium pseudocatenulatum in a sample from a subject using a metagenomics method as described herein.

The level of Bifidobacterium pseudocatenulatum may be compared to a reference value determined prior to administration of a composition as described herein. The reference value may be determined before a first administration of a composition as described herein, or after a first administration but before to a subsequent administration of a composition as described herein.

The method described herein are typically practiced outside of the human or animal body, e.g., on a sample that was previously obtained from the subject to be tested. Preferably, the sample is a faecal sample.

By way of example, the composition may increase the abundance of Bifidobacterium pseudocatenulatum by at least 1.5-, 2—, 3-, 4—, 5—, 10-, 50-, or 100-fold compared to, for example, the abundance of Bifidobacterium pseudocatenulatum prior to administration of the composition.

Bifidobacterium pseudocatenulatum has been reported to enhance bone mass density (BMD) by decreasing bone resorption and increasing bone formation (Fernández-Murga et al.; Bone; 2020; 141; 115580) and to reverse hyperleptinemia and restore leptin signals in obese mice (Agusti et al.; Mol. Neurobiol. 55 (6), 5337-5352).

The present invention provides a Bifidobacterium pseudocatenulatum microorganism for use in preventing and/or treating stunted growth in an infant or young child. Thus, the Bifidobacterium pseudocatenulatum may be provided as a probiotic, as defined herein.

Suitably, the Bifidobacterium pseudocatenulatum may be provided in a composition as defined herein. Said composition may be for use in preventing and/or treating stunted growth in an infant or young child. The composition may further comprise a prebiotic, such as a prebiotic as described herein.

Streptococcus thermophilus

The present invention may comprise the use of Streptococcus thermophilus as a probiotic or in a composition as described herein.

The present invention may further comprise a combination comprising the use of Streptococcus thermophilus as a probiotic or in a composition as described herein.

Streptococcus thermophilus is a gram-positive bacterium, and a fermentative facultative anaerobe, of the viridans group. It tests negative for cytochrome, oxidase, and catalase, and positive for alpha-hemolytic activity. It is non-motile and does not form endospores. It is also classified as a lactic acid bacterium.

A type strain of Streptococcus thermophilus is ATCC 19258. A reference genome for Streptococcus thermophilus is provided by GenBank assembly accession: GCA_903886475.1.

Suitably, the Streptococcus thermophilus may comprise a 16S rRNA gene sequence with a cutoff identity value of 99% and minimum query and target coverages of 80% when compared to the 16S rRNA sequence of ATCC 19258 and/or GCA_903886475.1 using BLASTn. Suitable comparisons may be performed using known methods, as described by Maturana and Cárdenasm (Front Microbiol. 2021; 660920), for example.

Suitably, the Streptococcus thermophilus may have ANI (average nucleotide identity) of at least 95%, a TETRA (tetranucleotide frequency) of at least 0.99, and/or an AAI (average amino acid identity) of at least 95% compared to ATCC 19258 and/or GCA_903886475 for an entire genome dataset. Suitably, the Streptococcus thermophilus microorganism may have ANI (average nucleotide identity) of at least 95%, a TETRA (tetranucleotide frequency) of at least 0.99, and an AAI (average amino acid identity) of at least 95% compared to ATCC 19258 and/or GCA_903886475 for an entire genome dataset. Suitable comparisons may be performed using known methods, as described by Maturana and Cárdenasm (as above), for example.

Suitably, a Streptococcus thermophilus may be identified using a metagenomics method. Suitable metagenomics methods may be performed using shotgun sequencing data, for example. Metagenomics methods may also advantageously enable estimation of organismal relative abundance. Suitable metogenomics methods are known in the art and include MetaPhlAn 3.0, for example (see Beghini et al.; eLife 2021; 10: e65088; https://huttenhower.sph.harvard.edu/metaphlan).

In some embodiments, the Streptococcus thermophilus is isolated from a human.

Suitably, to ‘promote Streptococcus thermophilus’ means to increase the absolute or relative numbers of Streptococcus thermophilus in the gut microbiota. For example, a prebiotic may assist or support the growth and/or survival of the microorganism. Alternatively, a probiotic composition for use in the invention may comprise Streptococcus thermophilus, and thereby increase number of Streptococcus thermophilus within the gut microbiota.

The abundance of Streptococcus thermophilus in the gut microbiota may be determined, for example, by assessing the relative abundance of Streptococcus thermophilus in a sample from a subject using a metagenomics method as described herein.

The level of Streptococcus thermophilus may be compared to a reference value determined prior to administration of a composition as described herein. The reference value may be determined before a first administration of a composition as described herein, or after a first administration but before to a subsequent administration of a composition as described herein.

By way of example, the composition may increase the abundance of Streptococcus thermophilus by at least 1.5-, 2-, 3-, 4-, 5-, 10-, 50-, or 100-fold compared to, for example, the abundance of Streptococcus thermophilus prior to administration of the composition.

### Prebiotic

Suitably, the composition may comprise a prebiotic.

The term “prebiotic” means non-digestible carbohydrates that beneficially affect the host by selectively stimulating the growth and/or the activity of healthy bacteria in the colon of humans (Gibson G R, et al. Nat Rev Gastroenterol Hepatol. 2017).

Suitably, the prebiotic is provided in the form of dietary fibers. For example, the dietary fibers may be prebiotic fibers.

Suitably, the prebiotic may be comprised in an ingredient, for example a dietary ingredient.

The ingredient may be selected from the group consisting of a human milk oligosaccharide (HMO), purified polysaccharide or purified oligosaccharide, a dietary fiber ingredient, a semi-purified food ingredient, a raw food ingredient, a food additive, a semi-purified or purified peptido-glycan.

Suitably, the prebiotic is a HMO.

Suitably, the prebiotic composition comprises a prebiotic (e.g., comprised in an ingredient or fibre) which promotes Bifidobacterium pseudocatenulatum in the gut microbiota. Suitably, the prebiotic composition comprises a prebiotic (e.g., comprised in an ingredient or fibre) which promotes Streptococcus thermophilus in the gut microbiota.

The prebiotic or composition may be provided as a fermented dairy product (e.g., yogurt). Fermented dairy product, such as yogurt, has been shown to increase S. thermophilus abundance in the gut microbiota (see e.g., Pasoli et al.; Nat Comm; 2000; 11(1); 2610; Oyarzun et al; Comput Struct Biotechnol J; 2022; 5(2); 1632-1641; Yazdi et al.; Journal of Functional Foods; 2022; 105089).

The composition may comprise oligosaccharide(s) (e.g., human milk oligosaccharides) and/or at least a fiber(s) and/or at least a precursor(s) thereof. The oligosaccharide and/or fiber and/or precursor thereof may be selected from the list comprising galacto-oligosaccharides (GOS), fructo-oligosaccharides (FOS), inulin, xylooligosaccharides (XOS), polydextrose and any combination thereof. They may be in an amount between 0 and 10% by weight of composition. In a particular embodiment, the nutritional composition can also contain at least one BMO (bovine milk oligosaccharide).

The present invention provides a prebiotic for use in preventing and/or treating stunted growth in an infant or young child. Suitably, the prebiotic promotes B. pseudocatenulatum in the gut microbiota of the infant or young child. Suitably, the prebiotic promotes S. thermophilus in the gut microbiota of the infant or young child.

Suitably, the prebiotic promotes B. pseudocatenulatum in the gut microbiota of the infant or young child in order to prevent and/or treat stunted growth in the infant or young child. Suitably, the prebiotic promotes S. thermophilus in the gut microbiota of the infant or young child in order to prevent and/or treat stunted growth in the infant or young child.

Suitably, the prebiotic may be provided in a composition as defined herein. Said composition may be for use in preventing and/or treating stunted growth in an infant or young child, for example by promoting B. pseudocatenulatum and/or S. thermophilus in the gut microbiota of the infant or young child. The composition may further comprise a probiotic, such as a B. pseudocatenulatum and/or S. thermophilus microorganism as described herein.

### Human Milk Oligosaccharide (HMO)

Suitably, the prebiotic may be a HMO.

The term “HMO” or “HMOs” refers to human milk oligosaccharide(s). These carbohydrates are highly resistant to enzymatic hydrolysis, indicating they may display essential functions not directly related to their caloric value. It has been especially illustrated they play a vital role in the early development of infants and young children, such as the maturation of the immune system. Many different kinds of HMOs are found in the human milk. Each individual oligosaccharide is based on a combination of glucose, galactose, sialic acid (N-acetylneuraminic acid), fucose and/or N-acetylglucosamine with many and varied linkages between them, thus accounting for the enormous number of different oligosaccharides in human milk-over 130 such structures have been identified so far. Almost all of them have a lactose moiety at their reducing end while sialic acid and/or fucose (when present) occupy the terminal position at the non-reducing ends. Depending on the presence of fucose and sialic acid in the oligosaccharide structure, the HMOs can be divided as non-fucosylated (neutral) or fucosylated (neutral) and sialylated (acidic) and non-sialylated molecules, respectively.

Suitably, the term “capable of metabolizing the HMO” may mean that the Bifidobacterium pseudocatenulatum encodes at least one CAZyme which is capable of utilizing the HMO. For example, the CAZyme may be capable of catalyzing the hydrolysis of a glycosidic bond within the HMO. Suitably, the Bifidobacterium pseudocatenulatum may encode at least one, at least two, at least three, at least four or at least five CAZymes that are capable of utilizing the HMO. Suitably, the term “capable of metabolizing the HMO” may mean that the HMO is capable of promoting growth and/or survival of the Bifidobacterium pseudocatenulatum (e.g., when added to an anaerobic culture of the Bifidobacterium pseudocatenulatum). Growth and/or survival of the B Bifidobacterium pseudocatenulatum may be determined by measuring the abundance of 16S rDNA—for example using PCR methods.

An HMO capable of promoting growth and/or survival of the Bifidobacterium pseudocatenulatum may increase the number of Bifidobacterium pseudocatenulatum in an anaerobic culture by at least 20%, at least 30%, at least 40%, at least 50%, at least 75% or at least 100% compared to the number of Bifidobacterium pseudocatenulatum in a control anaerobic culture which does not comprise the HMO. Suitably, HMO capable of promoting growth and/or survival of the Bifidobacterium pseudocatenulatum may increase the number of Bifidobacterium pseudocatenulatum in an anaerobic culture by a statistically significant amount (e.g. p-value <0.05 as determined by one-way ANOVA) compared to the number of Bifidobacterium pseudocatenulatum in a control anaerobic culture which does not comprise the HMO.

The preceding disclosures referencing Bifidobacterium pseudocatenulatum may be applied equally to Streptococcus thermophilus.

The expression “fucosylated oligosaccharide” refers to an oligosaccharide having a fucose residue. It has a neutral nature. Some examples are 2′-fucosyllactose (2-FL), 3-fucosyllactose (3-FL), difucosyllactose (DiFL), lacto-N-fucopentaose (e.g., lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof. Fucosylated oligosaccharides represents the largest fraction of human milk with 2′-FL constituting up to 30% of the total HMOs. Fucosylated oligosaccharides are thought to reduce the risk of infections and inflammations and to boost growth and metabolic activity of specific commensal microbes reducing inflammatory response.

The expression “N-acetylated oligosaccharide(s)” encompasses both “N-acetyl-lactosamine” and “oligosaccharide(s) containing N-acetyl-lactosamine”. They are neutral oligosaccharides having an N-acetyl-lactosamine residue. Suitable examples are LNT (lacto-N-tetraose), para-lacto-N-neohexaose (para-LNnH), LNnT (lacto-N-neotetraose), DSLNT (disialyllacto-N-tetraose), and any combinations thereof. Other examples are lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose.

The expressions “at least one fucosylated oligosaccharide” and “at least one N-acetylated oligosaccharide” should be understood as “at least one type of fucosylated oligosaccharide” and “at least one type of N-acetylated oligosaccharide”.

The term “sialylated oligosaccharide” refers to an oligosaccharide having a charged sialic acid residue. It has an acidic nature. Some examples are 3′-sialyllactose (3-SL), 6′-sialyllactose (6-SL), sialyllacto-N-tetraose (Lst—e.g., Lst-a, Lst-b or Lst-c).

The HMO may be a fucosylated oligosaccharide (i.e. an oligosaccharide having a fucose residue; e.g. 2′ fucosyllactose (2-FL), 3-fucosyllactose (3-FL), difucosyllactose (DiFL), lacto-N-fucopentaose (e.g. lacto-N-fucopentaose I, lacto-N-fucopentaose II, lacto-N-fucopentaose III, lacto-N-fucopentaose V), lacto-N-fucohexaose, lacto-N-difucohexaose I, fucosyllacto-N-hexaose, fucosyllacto-N-neohexaose, difucosyllacto-N-hexaose I, difucosyllacto-N-neohexaose II and any combination thereof), an N-acetylated oligosaccharide (e.g. LNT (lacto-N-tetraose), para-lacto-N-neohexaose (para-LNnH), LNnT (lacto-N-neotetraose), DSLNT (disialyllacto-N-tetraose), lacto-N-hexaose, lacto-N-neohexaose, para-lacto-N-hexaose, para-lacto-N-neohexaose, lacto-N-octaose, lacto-N-neooctaose, iso-lacto-N-octaose, para-lacto-N-octaose and lacto-N-decaose and any combinations thereof) and/or a sialylated oligosaccharide (e.g. 3′-sialyllactose (3-SL), 6′-sialyllactose (6-SL), or Lst (sialyllacto-N-tetraose), Lst-a, Lst-b or Lst-c)).

Suitably, the prebiotic may comprise a HMO may be selected from the group consisting of 2′-FL, di-FL, 6′-SL and LNnT, and any combination thereof. The HMOs may be a combination of 2′-FL and di-FL. The HMOs may be a combination of 6′-SL and LNnT.

The HMO(s) may be provided in combination with galacto-oligosaccharides (GOS).

The prebiotic may comprise at least one prebiotic oligosaccharide selected from the group consisting of: 2′-O-fucosyllactose (2′FL), 3′-O-fucosyllactose (3FL), lactodifucotetraose/difucosyllactose (DFL), 3′-O-sialyllactose (3-SL), 6′-O-sialyllactose (6-SL), lacto-N-tetraose (LNT) and lacto-N-neotetraose (LNnT); and any combination thereof.

The prebiotic may comprise at least on prebiotic oligosaccharide selected from the group consisting of: 3FL, 3′SL, LNnT, lacto-N-fucopentaose (LNFP I), LNFP II, LNFP III, sialyl-lacto-N-tetraose (LST) b, LSTc, disialyllacto-N-tetraose (DSLNT), fucosyllacto-N-hexaose (FLNH), difucosyllacto-N-hexaose (DFLNH) and disialyllacto-N-hexaose (DSLNH); and any combination thereof.

The prebiotic may comprise 34 wt % to 85 wt % of 2′-FL, 10 wt % to 40 wt % of LNT, 4 wt % to 14 wt % of DFL and 9 wt % to 31 wt % of 3-SL and 6-SL combined.

In some embodiments, the prebiotic comprises
26 wt % to 65 wt % of 2′-FL, preferably 32 wt % to 54 wt %;
10 wt % to 40 wt % of LNT, preferably 11 wt % to 20 wt %;
4 wt % to 14 wt % of DFL, preferably 4 wt % to 8 wt %;
9 wt % to 31 wt % of 3′-SL and 6′-SL combined, preferably 8 wt % to 22 wt %; and
12 wt % to 38 wt % of 3-FL, preferably 17 wt % to 31 wt %.

The prebiotic may comprise between 0.001 g/L to 12 g/L of 2′-FL, preferably between 0.002 g/L to 10 g/L of 2′-FL, more preferably between 0.005 g/L to 5 g/L of 2′-FL.

The prebiotic may comprise between 0.001 g/L to 5 g/L of DFL, preferably between 0.002 g/L to 4 g/L of DFL, more preferably between 4 g/L to 3 g/L of DFL.

The prebiotic may comprise between 0.01 g/L to 6 g/L of LNT, preferably between 0.025 g/L to 5 g/L of LNT, more preferably between 0.05 g/L to 1 g/L of LNT.

The prebiotic may comprise between 0.001 g/L to 2 g/L of 6′-SL, preferably between 0.002 g/L to 1.5 g/L of 6′-SL, more preferably between 0.005 g/L to 1 g/L of 6′-SL.

The prebiotic may comprise between 0.01 g/L to 2 g/L of 3′-SL, preferably between 0.025 g/L to 1.5 g/L of 3′-SL, more preferably between 0.05 g/L to 1 g/L of 3′-SL.

The prebiotic may comprise between 0.01 g/L to 7 g/L of 3-FL, preferably between 0.025 g/L to 6 g/L of 3-FL, more preferably between 0.05 g/L to 5 g/L of 3-FL.

Suitably, the mixture of oligosaccharides comprises or consists of 2′-fucosyllactose (2′FL), difucosyllactose (diFL), lacto-N-tetraose (LNT) and lacto-N-neotetraose (LNnT). In some embodiments, the mixture of oligosaccharides comprises or consists of 3′-sialyllactose (3′-SL), 6′-sialyllactose (6′-SL), 2′-fucosyllactose (2′FL), difucosyllactose (diFL), lacto-N-tetraose (LNT) and lacto-N-neotetraose (LNnT).

In some embodiments, the mixture of oligosaccharides comprises:
10 to 35 wt %, preferably 10 to 30 wt %, more preferably 10 to 25 wt %, with respect to the total weight of the oligosaccharide mixture, of at least one sialylated oligosaccharide;
30 to 80 wt %, preferably 40 to 80 wt %, more preferably 50 to 70 wt %, with respect to the total weight of the oligosaccharide mixture, of at least one fucosylated oligosaccharide; and/or
10 to 35 wt %, preferably 15 to 30 wt %, more preferably 15 to 20 wt %, with respect to the total weight of the oligosaccharide mixture, of at least one N-acetylated oligosaccharide.

The present invention provides a HMO or combination of HMOs for use in preventing and/or treating stunted growth in an infant or young child. Suitably, the HMO(s) promotes B. pseudocatenulatum and/or S. thermophilus in the gut microbiota of the infant or young child.

Suitably, the HMO(s) may be provided in a composition as defined herein. Said composition may be for use in preventing and/or treating stunted growth in an infant or young child. The composition may further comprise a probiotic, such as a B. pseudocatenulatum microorganism as described herein.

HMOs have been shown to increase the abundance of B. pseudocatenulatum in infant microbiota (e.g., Cheema et al.; Int J Mol Sci; 2022; 23 (5): 2804 and the present Examples). Levels of different HMOs in breast milk have also been reported to have associations with infant growth including length/height (Samuel T M, et al. Sci Rep. 2022).

### Microbiota and Microbiome

The “gut microbiota” may refer to the composition of microorganisms (including bacteria, archaea and fungi) that live in the digestive tract.

The term “gut microbiome” may encompass both the “gut microbiota” and their “theatre of activity”, which may include their structural elements (nucleic acids, proteins, lipids, polysaccharides), metabolites (signalling molecules, toxins, organic, and inorganic molecules), and molecules produced by coexisting hosts and structured by the surrounding environmental conditions (see e.g., Berg, G., et al., 2020. Microbiome, 8 (1), pp. 1-22).

In the present invention, the term “gut microbiome” may therefore be used interchangeably with the term “gut microbiota”.

### Method

The present invention further provides a method for predicting or assessing if an infant or young child is at risk of stunted growth, said method comprising determining the level of Bifidobacterium pseudocatenulatum in one or more samples obtained from the infant or young child.

Without wishing to be bound by theory, the inventors have determined that an infant or young child with lower levels of Bifidobacterium pseudocatenulatum (e.g., abundance and/or activity) in their microbiome may have an increased likelihood of stunted growth.

The level of Bifidobacterium pseudocatenulatum may be compared to a reference value, wherein the comparison is indicative of the predicted risk of stunted growth for the infant or small child. The term reference level is synonymous with ‘control level’ and broadly includes data that the skilled person would use to facilitate the accurate interpretation of technical data.

The reference value may be based on a value (e.g., an average) of Bifidobacterium pseudocatenulatum in a population of infants and/or young children who are known to be at risk of stunted growth or to have stunted growth. The reference value may be based on a value (e.g., an average) of Bifidobacterium pseudocatenulatum in a population of infants and/or young children who are known not to be at risk of stunted growth or who do not have stunted growth. The reference value may be based on a value (e.g., an average) of Bifidobacterium pseudocatenulatum in a population of infants and/or young children who are known not to be at risk of stunted growth or to have stunted growth.

The reference level may be age matched with the test sample.

Suitably, the infant or young child may be from about 1 to 60 months, about 1 to 48 months, about 2 to 60 months, about 2 to 48 months, about 2 to 36 months, or about 4 to 36 months, about 6 to 36 months of age or about 6 to 24 months of age.

Preferably, the infant or young child may be at least 10 months of age.

For example, the infant or young child may be at least about 10 months, at least about 12 months, at least about 14 months, at least about 16 months, at least about 20 months, or at least about 24 months.

Preferably, the infant or young child may be from about 10 to about 48 months of age, about 10 to about 36 months of age, about 10 to about 24 months of age, about 10 to about 18 months of age.

The present method is typically practiced outside of the human or animal body, e.g. on a sample that was previously obtained from the subject to be tested. Preferably, the sample is a faecal sample.

Suitably, the present method provides that a difference in the level of Bifidobacterium pseudocatenulatum in the test sample compared to the reference level is indicative of the risk of stunted growth. Suitably, the present method may provide that a difference in the level of Bifidobacterium pseudocatenulatum in the test sample compared to the reference level is indicative of an increased risk of stunted growth.

For example, a 1.1, 1.5, 2, 3, 4, 5, 10, 50, or 100-fold difference between the levels determined in the test sample and the reference level may be indicative of an increased risk of stunted growth.

Suitably, a reduced level of Bifidobacterium pseudocatenulatum is associated with an increased risk of stunted growth. Suitably, an infant or young child with reduced levels of Bifidobacterium pseudocatenulatum is identified as at risk of stunted growth.

For example, a 1.1, 1.5, 2, 3, 4, 5, 10, 50, or 100-fold lower level of Bifidobacterium pseudocatenulatum determined in the test sample compared to the reference level may be indicative of an increased risk of stunted growth.

Suitably, the method further comprises combining the level of Bifidobacterium pseudocatenulatum with one or more anthropometric measures.

Suitably, an infant or young child determined to be at risk of stunted growth using the present method may be treated with a composition to reduce the risk of occurrence of stunted growth and/or prevent stunted growth according to the present invention.

Suitably, the present methods may comprise determining the level of Streptococcus thermophilus as an alternative to Bifidobacterium pseudocatenulatum.Suitably, the present methods may comprise determining the level of Bifidobacterium pseudocatenulatum and Streptococcus thermophilus.

The present method may be performed on one or more samples obtained from the subject. For example, the method may be performed using a first sample obtained at a given time point and a second sample obtained following a time interval after the first sample was obtained. The method may be performed more than once, on samples obtained from the same subject over a time period. For example, samples may be obtained repeatedly once per month, once a year, or once every two years.

### Examples

The invention will now be further described by way of examples, which are meant to serve to assist the skilled person in carrying out the invention and are not intended in any way to limit the scope of the invention.

### Example 1: Microbiota and Health Study

The microhealth cohort is described in Vidal et al. (https://www.medrxiv.org/content/10.1101/19000505v1) and registered at clinicaltrials.gov as NCT02361164.

Briefly, for n=220 infant or young children, the following information was collected: Anthropometry, Diarrhea and Acute Respiratory Infections (ARI); Medication, including antibiotic use; Breastfeeding status, weaning foods; Nasopharyngeal and stool pathogens and microbiota profiles; Secretor status (FUT2, FUT3). Stool samples for microbiota analysis were collected at birth, 2 months, 6 months, 10 months, 15 months, 18 months, and 24 months. Growth outcomes were noted at birth, 2 months, 4 months, 6 months, 8 months, 10 months, 12 months, 15 months, 18 months and 24 months. See FIG. 1 for an overview.

### Example 2: Definition of Reference and Undergrowth Populations

Based on WHO guidelines undergrowth was defined by (i) dynamic changes or (ii) static outcome.

For dynamic changes, change in length-for-age z-score (LAZ) was assessed over time (6-24 months). We identified three patterns—not much change in LAZ score over time (negligible slopes), decline in LAZ scores over time (negative slopes), and improvement in LAZ scores over time (positive slopes). Infants with a change in LAZ scores over time, calculated as slopes, with <−0.0485434516523868 were called as ‘undergrowth’ (n=48). Infants with change in LAZ scores over time, calculated as slopes, with >0.00495716034271725 were called as the reference population (n=48). The microbiota of infants in first quartile on change of length-for-age z-score (LAZ) from 6-24m, having faltering growth, were compared with infants in fourth quartile, having improving growth.

For static outcome, the LAZ score at 24 months of age was assessed, and defined length_for_age_24m <−2 as ‘undergrowth’, and length_for_age_24m >=−2 as the reference population.

### Example 3: Differences in Microbiota Between Undergrowth and Reference Populations

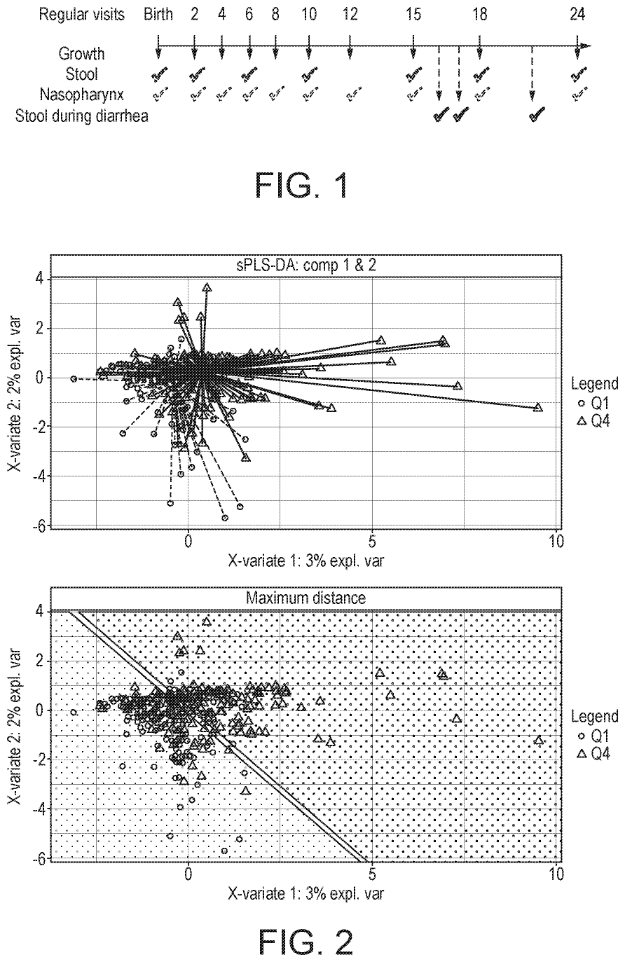

For dynamic changes the microbiota data was only taken for samples corresponding to 6-24 months. First, we filtered out any bacteria species with less than mean 0.01% relative abundance. Thus, from about 750 species, 168 species remained after this step. Next, we removed features with near zero variance using the nearZeroVar function of the mixOmics Rpackage, with these parameters-freqCut=95/05, uniqueCut=20. Following this step, 78 species remained (see FIG. 2).

To determine if there are overall microbiota differences between the two groups, ‘Reference’ vs. ‘Undergrowth’, as defined above, we used a Machine Learning algorithm-sPLS-DA (Lê Cao K A, et al. BMC Bioinformatics. 2011). The feature selection to find differentiating bacteria was performed in an M-fold mode of 10-folds, 10-repeats with the centroids distance measure, keeping a balanced error rate between the two classes. To identify time intervals of differentially abundant features in metagenomic longitudinal studies we used an R-package-MetaLonDA (Metwally A A, et al. Microbiome. 2018). We ran MetaLonDA in a “screening mode” with 100 permutations. Hits thus identified were confirmed by rerunning MetaLonDA with the recommended 1000 permutations.

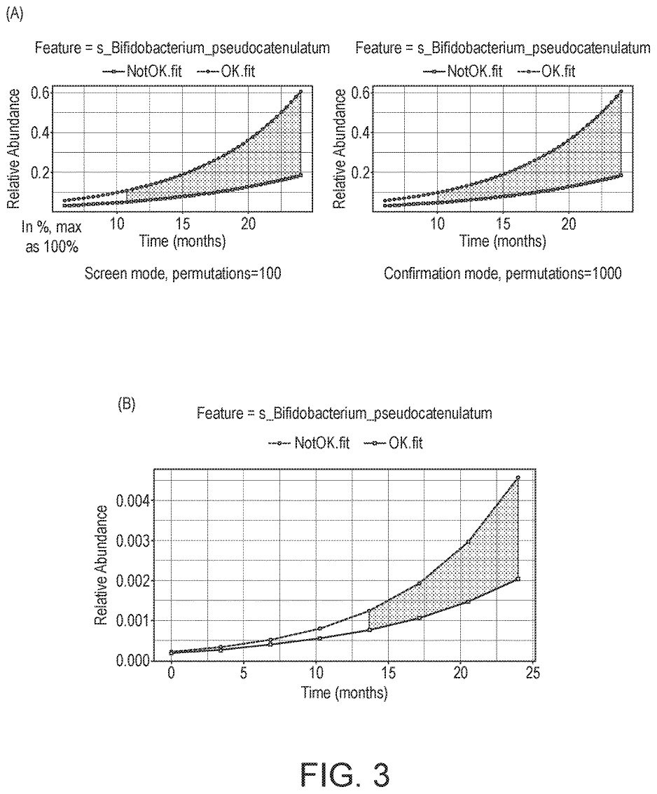

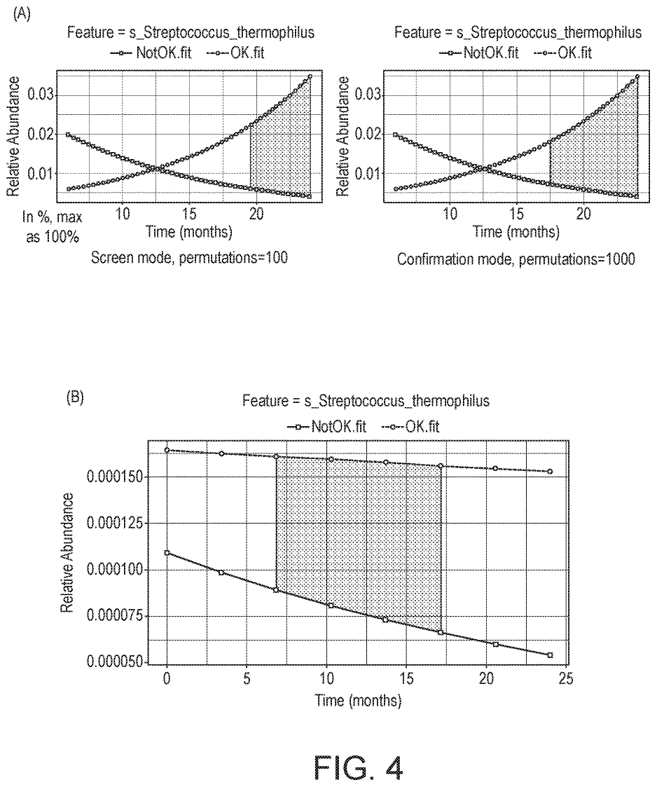

For static outcomes, similar technical methods as above were performed to identify bacteria that were found to be significantly different across time between the two groups (length_for_age_24m: <−2 as ‘Undergrowth’; >=−2 as ‘Reference’) (see FIGS. 3 and 4).

Common hits were identified by multiple algorithms, associated with dynamic changes (6-24m) of length-for-age zscore (LAZ) & static measure (at 24m) of laz<−2 or >=−2. On multiple lines of evidence, B. pseudocatenulatum and S. thermophilus were identified as a bacterial signature of inadequate height profile up to 24 months.

### Example 4: Effect of Galacto-Oligosaccharide and HMOs on B. pseudocatenulatum in Infant Microbiome

Samples were tested with combinations of galacto-oligosaccharide (GOS), HMO1 (2′ FL and diFL) and/or HMO2 (LNnT and 6′ SL) using ex vivo D-SIFR® technology simulating the gut microbiota of three 3 year-old toddlers.

Treatments with GOS, HMO1 and/or HMO2 were found to have profound bifidogenic effects, including promoting levels of B. pseudocatenulatum. For example, treatment with HMOs (+/−GOS) increased B. pseudocatenulatum levels by at least 2-fold.

### Materials & Methods

Faecal samples were collected according to a procedure approved by Ethics Committee of the University Hospital Ghent.

Colonic fermentation of the test products by gut microbioata in the faecal samples was assessed at 24 hours post-inoculation.

For the statistical evaluation of the treatment effects on fundamental fermentation parameters, cell counts, microbial diversity and microbial composition (phylum level) across samples from 3 infants, a repeated measures ANOVA analysis was performed (~ based on paired t-testing, thus accounting for fact that values are compared between samples of a given donor). The statistical significance of the potential treatment effects was determined via Benjamini-Hochberg post hoc testing

For quantitative shallow shotgun sequencing, upon DNA extraction, standardized Illumina library preparation was performed followed by 3M total DNA sequencing.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

### Embodiments

Various preferred features and embodiments of the present invention will now be described with reference to the following numbered paragraphs (paras).

1. A method for identifying an infant or young child at risk of stunted growth, wherein said method comprises determining the abundance of Bifidobacterium pseudocatenulatum and/or Streptococcus thermophilus in one or more samples obtained from the infant.

2. The method according to para 1, wherein an infant or young child with reduced levels of B. pseudocatenulatum is identified as at risk of stunted growth.

3. The method according to para 1 or 2, which further comprises determining the abundance of Bifidobacterium pseudocatenulatum and Streptococcus thermophilus in one or more samples obtained from the infant.

4. The method according to para 3, wherein an infant or young child with reduced levels of S. thermophilus is identified as at risk of stunted growth.

5. The method according to any preceding para wherein the stunted growth is associated with malnourishment.

6. The method according to any preceding para wherein the stunted growth is stunted height or length.

7. The method according to para 6 wherein the stunted height or length is defined as a reduced length-for-age z-score (LAZ) or a reduced LAZ over time or a reduced height-for-age z-score (HAZ) or a reduced HAZ over time.

8. A composition for use in preventing and/or treating stunted growth in an infant or young child, wherein the composition promotes B. pseudocatenulatum and/or Streptococcus thermophilus in the gut microbiota of the infant or young child.

9. The composition for use according to para 8, wherein the composition promotes a B. pseudocatenulatum in the gut microbiota of the infant or young child; optionally wherein the composition comprises a B. pseudocatenulatum microorganism.

10. The composition for use according to para 8 or 9, wherein the composition promotes S. thermophilus in the gut microbiota of the infant or young child; optionally wherein the composition comprises a S. thermophilus microorganism.

11. The composition for use according to para 9 or 10, wherein the composition is administered in combination with a prebiotic.

12. The composition for use according to para 8, wherein the composition comprises a prebiotic.

13. The composition for use according to para 12, wherein the composition is administered in combination with a B. pseudocatenulatum and/or Streptococcus thermophilus microorganism.

14. A combination of a B. pseudocatenulatum and/or Streptococcus thermophilus microorganism and a prebiotic for use in preventing and/or treating stunted growth in an infant or young child.

15. The composition or combination for use according to any of paras 11 to 14, wherein the prebiotic is in the form of a dietary or nutritional composition.

16. The composition or combination for use according to any of paras 11 to 15 wherein the prebiotic comprises a human milk oligosaccharide (HMO).

17. A human milk oligosaccharide (HMO) or a combination of HMOs for use in preventing and/or treating stunted growth in an infant or young child, wherein the HMO or combination of HMOs promote B. pseudocatenulatum and/or Streptococcus thermophilus in the gut microbiota of the infant or young child.

18. The composition or combination for use according to para 16 or the HMO or combination of HMOs for use according to para 17 wherein the HMO is selected from the group consisting of 2′-FL, 3-FL, di-FL, 3′-SL, 6′-SL, LNT and LNnT, and any combination thereof; suitably wherein the HMOs are (i) a combination of 2′-FL and di-FL or (ii) a combination of 6′-SL and LNnT.

19. A B. pseudocatenulatum microorganism for use in preventing and/or treating stunted growth in an infant or young child.

20. The composition, combination, HMO or B. pseudocatenulatum and/or Streptococcus thermophilus microorganism for use according to any of paras 8 to 19 wherein the infant or young child has been determined to have reduced levels of B. pseudocatenulatum in the gut microbiota.

21. The composition, combination, HMO or B. pseudocatenulatum and/or Streptococcus thermophilus microorganism for use according to any of paras 8 to 19 wherein the infant or young child has been determined to be at risk of stunted growth by the method of any of paras 1 to 7.

22. The composition, combination, HMO or B. pseudocatenulatum and/or Streptococcus thermophilus microorganism for use according to any of paras 8 to 21 wherein the stunted growth is associated with malnourishment.

23. The composition, combination, HMO or B. pseudocatenulatum and/or Streptococcus thermophilus microorganism for use according to any of paras 8 to 22 wherein the stunted growth is stunted height or length.

24. The composition, combination, HMO or B. pseudocatenulatum and/or Streptococcus thermophilus microorganism for use according to para 23 wherein the stunted height or length is defined as a reduced length-for-age z-score (LAZ) or a reduced LAZ over time or a reduced height-for-age z-score (HAZ) or a reduced HAZ over time.

25 The composition, combination, HMO or B. pseudocatenulatum and/or Streptococcus thermophilus microorganism for use according to any of paras 8 to 24 wherein bone development and/or bone strength is enhanced in the subject.

26. A method of preventing and/or treating stunted growth in an infant or young child, said method comprising administering a composition which promotes B. pseudocatenulatum and/or Streptococcus thermophilus in the gut microbiota to the infant or young child.

27 Use of a composition for modulating the abundance of B. pseudocatenulatum and/or Streptococcus thermophilus in the gut of an infant or young child.

28. The use according to para 27 wherein the composition is a composition as defined in any of paras 6 to 18.

29. Use of a HMO or combination of HMOs for modulating the abundance of B. pseudocatenulatum and/or Streptococcus thermophilus in the gut of an infant or young child.

31. The method, composition for use, combination for use or use according to any preceding para wherein in the infant or young child is less than about 60 months of age, suitably less than 36 months of age, less than 24 months of age, suitably from about 6 to about 24 months of age.

32. The method, composition for use, combination for use or use according to any preceding para wherein the microorganism is B. pseudocatenulatum.

33. A probiotic composition comprising B. pseudocatenulatum.

34. The probiotic composition according to para 31, wherein the probiotic composition further comprises S. thermophilus.

35. A synbiotic composition comprising B. pseudocatenulatum and a prebiotic.

36. The synbiotic composition according to para 33 wherein the prebiotic promotes B. pseudocatenulatum in the gut microbiota of an infant or young child.

37. The synbiotic composition according to para 35 or 36 wherein the prebiotic is as defined in any of paras 15 to 18.

1. A method for identifying an infant or young child at risk of stunted growth, wherein the method comprises determining an abundance of Bifidobacterium pseudocatenulatum and/or Streptococcus thermophilus in one or more samples obtained from the infant or young child.

2. The method according to claim 1, wherein [an] the infant or young child has reduced levels of B. pseudocatenulatum and is identified as at risk of stunted growth.

3. The method according to claim 1, wherein the stunted growth is at least one of stunted height or length.

4. A method of preventing and/or treating stunted growth in an infant or young child, the method comprising administering a composition to the infant or young child, wherein the composition promotes B. pseudocatenulatum and/or Streptococcus thermophilus in the gut microbiota of the infant or young child.

5. The method according to claim 4, wherein the composition promotes B. pseudocatenulatum in the gut microbiota of the infant or young child.

6. The method according to claim 4, wherein the composition comprises a prebiotic.

7. The method according to claim 4, wherein the composition comprises a combination of a B. pseudocatenulatum and/or Streptococcus thermophilus microorganism and a prebiotic.

8. The method according to claim 4, wherein the composition is a dietary or nutritional composition.

9. The method according to claim 6, wherein the prebiotic comprises a human milk oligosaccharide (HMO).

10. The method according to claim 9, wherein the HMO promotes B. pseudocatenulatum and/or Streptococcus thermophilus in the gut microbiota of the infant or young child.

11. The method according to claim 4, wherein the composition comprises a B. pseudocatenulatum and/or Streptococcus thermophilus microorganism.

12. The method according to claim 2 further comprising administering a composition to the infant or young child identified as at risk of stunted growth, wherein the composition comprises at least one of a prebiotic or a B. pseudocatenulatum and/or Streptococcus thermophilus microorganism.

13. The method according to claim 4 wherein the stunted growth is at least one of stunted height or stunted length.

14. The method according to claim 4 wherein bone development and/or bone strength is enhanced in the infant or young child.

15. The method of claim 4, wherein the composition is a synbiotic composition comprising B. pseudocatenulatum and/or Streptococcus thermophilus and a prebiotic, wherein the prebiotic is a HMO which promotes B. pseudocatenulatum and/or Streptococcus thermophilus in the gut microbiota of the infant or young child.

16. The method according to claim 3, wherein the stunted height or stunted length is defined as a reduced length-for-age z-score (LAZ) or a reduced LAZ over time or a reduced height-for-age z-score (HAZ) or a reduced HAZ over time.

17. The method according to claim 5, wherein the composition comprises a B. pseudocatenulatum microorganism.

18. The method according to claim 13, wherein the stunted height or stunted length is defined as a reduced length-for-age z-score (LAZ) or a reduced LAZ over time or a reduced height-for-age z-score (HAZ) or a reduced HAZ over time.
